(19) United States
(12) Patent Application Publication
(10) Pub. No.: US 2026/0355102 A1
(43) Pub. Date: Nov. 12, 2026

(54) NOVEL USES OF A PHAEODACTYLUM TRICORNUTUM EXTRACT

(71) Applicant: MICROPHYT, Baillargues (FR)

(72) Inventors: Cyrielle HOUDIN, Baillargues (FR); Rémi PRADELLES, Baillargues (FR)

(73) Assignee: MICROPHYT, Baillargues (FR)

(21) Appl. No.: 19/140,537

(22) PCT Filed: Dec. 19, 2023
(86) PCT No.: PCT/FR2023/052047

§ 371 Date: Jun. 18, 2025

(30) Foreign Application Priority Data
Dec. 19, 2022 (FR) FR2213874

(51) Int. Cl. A61K 8/9711; A61Q 19/00; A61Q 19/08

(52) U.S. Cl. CPC A61K 8/9711; A61Q 19/001; A61Q 19/08; A61K 2800/49; A61K 2800/522; A61K 2800/92

(57) ABSTRACT

The present invention relates in particular to the cosmetic use of an extract of Phaeodactylum tricornutum to maintain and/or increase the volume of the skin and/or lips, and/or plump the skin, advantageously the skin of the face, and/or the lips, and/or fill in hollow areas of the face. The extract is effective for plumping skin and lips.

### INVENTION DOMAIN

The invention is related to new uses of an extract of Phaeodactylum tricornutum, in particular in the cosmetic and the nutraceutical field.

### BACKGROUND

The skin is a major organ, protecting the body from environmental attacks such as cold, wind and pollution, as well as from toxins and bacterial attacks. It is a complex structure made up of three successive layers, the dermis, the dermis and the hypodermis. The epidermis is the outermost layer. The dermis or corion lies beneath the epidermis and is made up of 2 layers, the reticular layer in contact with the hypodermis and the papillary layer in contact with the epidermis. The hypodermis is the deepest layer, mainly made up of adipocytes and blood vessels.

It is the dermis that gives to skin its suppleness and elasticity. The dermis contains the elastin and collagen fibers that give the skin its properties. But the dermis also includes connective tissue. This connective tissue, also known as fundamental substance, is mainly made up of hyaluronic acid. It is this fundamental substance that gives the skin its volume. Hyaluronic acid is a cross-linked glycosaminoglycan (GAG) polymer present in all human connective, epithelial and nervous tissues. It is synthesized by fibroblasts. It consists of D-glucuronic acid and D-N-acetylglucosamine linked by β-1,4 and β-1,3 glycosidic bonds. Its role in the skin is one of tissue filling and cohesion.

One of the causes of sagging skin with age is a reduction in the quantity of collagen and elastin fibers. Reduced cell renewal, particularly of the fibroblasts present in the extra-cellular matrix (ECM), is also one of the causes of sagging skin. Skin loses its elasticity and firmness, and wrinkles appear. Factors such as smoking, an unbalanced diet, pollution or UV rays are aggravating factors and accelerate these signs of aging. Hormonal changes or weight loss can also lead to visible sagging of the skin. However, while loss of skin firmness and elasticity are relatively easy to understand, loss of volume with age is trickier to describe.

Loss of volume, however, is a separate cause from loss of skin firmness and elasticity, which also contributes to skin aging. This loss of volume leads to sagging of the skin, particularly of the facial oval, visibly changing the face's shape and expression. A young person's plump cheeks and cheekbones become flat, the mouth becomes droopy and the face hollows. This is due to a reduction in certain proteins of the fundamental substance, in particular the production of hyaluronic acid.

To counteract this loss of volume, the best-known active ingredient is hyaluronic acid. Other invasive techniques, such as hyaluronic acid injections, do exist, but only provide a temporary effect.

There is therefore a need to develop alternative solutions, which are also natural and non-invasive, aimed at stimulating and/or increasing hyaluronic acid synthesis in the fundamental substance, for a plumping, volumizing effect on the skin.

Unexpectedly, the Applicant discovered that an extract of P. tricornutum solved the technical problem posed, by its ability to increase skin volume, and therefore plump it, by increasing the amount of hyaluronic acid in the fundamental substance.

One advantage of the extract according to the invention is that it is a perfectly natural extract, non-irritating to the skin. Another advantage is that it is very easily produced on an industrial scale, via biomass production in photobioreactors under controlled conditions. It can also be easily incorporated into cosmetic formulations. The extract is chlorophyll-free and obtained using a biobased solvent. In addition, the plumping effect of the extract is achieved without invasive intervention or microsurgery, providing an undeniable advantage over injection techniques aimed at the same effect, which lead to hematoma and redness.

The extract according to the invention is extracted from the biomass of the microalga P. tricornutum. Phaeodactylum tricornutum is a diatom belonging to the genus Phaeodactylum and the family Phaeodactylaceae, present in 3 distinct morphotypes (fusiform, oval, triradiate) found in many oceanic regions of the world, including northern Europe, Oceania and the Atlantic.

The use of extracts of the microalga P. tricornutum in cosmetics are known, in particular an aqueous extract of P. tricornutum, to reduce skin redness, relieve sensitive skin or improve the cutaneous microbiota of sensitive skin (WO2020178213), an extract of P. tricornutum as a depigmenting active agent to attenuate or remove pigmentation spots on the skin or lighten the complexion (FR2913336) or a P. tricornutum extract to improve skin firmness and elasticity and reduce wrinkles (WO02/080876).

However, none of these documents describes the use of a P. tricornutum extract to maintain and/or increase skin volume, or an effect on maintaining and/or increasing the amount of hyaluronic acid in the fundamental substance.

To the best of the Applicant's knowledge, the use of any P. tricornutum extract to maintain and/or increase the volume of skin and/or lip has never been described.

### BRIEF DESCRIPTION OF THE INVENTION

A first object of the invention relates to the cosmetic use of a P. tricornutum extract to maintain and/or increase the volume of the skin and/or lips.

Another object relates to a method of cosmetic care to maintain and/or increase the volume of the skin and/or lips, and/or to plump skin and/or lips, and/or to fill hollow areas of the face comprising the oral administration or the topical application, preferably the topical application, of an extract of P. tricornutum as defined according to the invention or of a cosmetic composition comprising it.

A final object relates to an extract of P. tricornutum or a dermatological or pharmaceutical composition comprising it for use in promoting healing of the skin, in particular of persons with damaged skin and/or damaged lips, wounds, lesions and/or superficial wounds.

### DETAILED DESCRIPTION

A first object therefore concerns the cosmetic use of an extract of Phaeodactylum tricornutum to maintain and/or increase the volume of the skin and/or the lips.

According to a particular mode, the extract of P. tricornutum is used according to the invention to plump skin, advantageously facial skin, and/or lips, and/or fill hollow areas of the face.

By “cosmetic use” it is meant a non-therapeutic use, i.e. neither pharmaceutical nor dermatological according to a specialist in the field, a dermatologist, i.e. not intended to treat a skin or lips which require a therapeutic treatment. Thus, said use is advantageously intended for healthy skin and/or healthy lips. By “healthy skin or healthy lip” it is meant any area of skin or lip qualified as non-pathological by a dermatologist, i.e. an area of skin or lip free of, for example, injury, redness, infection, scarring, allergy, wound, disease, eczema, inflammation, injury, acne and/or dermatitis. Said use is thus not intended to treat a skin and/or lips presenting a pathology, but to prevent and/or improve aesthetic disorders induced by a loss of volume in the skin and/or lips.

The use of the extract of P. tricornutum according to the invention can be through oral administration or by topical application.

Preferentially, this is a topical application. By “topical application” it is meant the application onto at least one area of the skin or lips, or the spraying onto the surface of at least one area of the skin, of the extract according to the invention or of a composition comprising it.

The extract of P. tricornutum used according to the invention or the composition comprising it is topically acceptable, i.e. it does not induce an allergic reaction, is not toxic or irritating. The extract according to the invention or the composition comprising it can be applied or sprayed onto at least one area of skin chosen from any area of the face and/or body, and/or on the lips. “Any area of the body” means any area of the arms, legs, thighs, back, torso, chest, neck, feet and/or hands, and/or buttocks. “Any area of the face” means any area selected from the cheeks, cheekbones, forehead, nose contour, nasolabial fold, temples, eye contour, chin and/or mouth contour. The extract of P. tricornutum used according to the invention or the composition comprising it is advantageously applied onto any area of the face, in particular the cheeks, cheekbones, eye contour and lips, to plump them and restore their volume.

On the face, volume loss tends to affect the central region of the face, with flattening of the cheekbones and sagging of the cheeks, leading to the appearance of dark circles, the valley of tears and the nasolabial fold. The loss of volume also affects the forehead, which loses its convexity and brings out the relief of the superciliary arches, as well as the temples, which deepen from the age of forty onwards.

In particular, the extract of P. tricornutum according to the invention is intended to plump and restore volume to the skin and/or lips of people with hollowed facial areas, particularly in the cheeks, cheekbones, eye contours and lips. Loss of facial skin volume can occur with ageing, but also in people who have lost weight. Cheeks, cheekbones and dark circles under the eyes tend to deepen and lose volume. Particularly in the case of people with facial hollows, the aim is to fill these areas to reshape the face and add volume to the look, for a more youthful, less tired appearance.

“Maintaining and/or increasing the volume of the skin and/or lips” further means maintaining and/or increasing the amount of hyaluronic acid in the fundamental substance of the skin and/or lips, advantageously the facial skin and/or lips. The term “hyaluronic acid quantity” refers to the quantity of hyaluronic acid proteins. This clearly distinguishes the volumizing effect of the extract used according to the invention on the skin or lips, i.e. a plumping effect, from the effects on skin firmness and/or elasticity described above, which act on other skin mechanisms and proteins.

By “increasing the volume of the skin and/or lips”, it is also meant, according to the invention, improving the remodeling of the skin and/or lips under the plumping effect of the extract according to the invention.

Extract of P. tricornutum

### Mode of Production of Microalgae

The extract according to the invention is obtained from the microalga P. tricornutum cultivated under controlled conditions within adapted systems such as open culture ponds of “race-ways” or “open ponds” type, or closed systems such as photobioreactor. The photobioreactors used can be of any existing type, such as horizontal tubular photobioreactors, vertical photobioreactors such as “green wall panel” systems, flat photobioreactors or column photobioreactors. Preferably, the biomass is produced in a closed photobioreactor-type culture system.

Microalgae are cultivated in batch, fed-batch, continuous, semi-continuous, turbidostat or chemostat modes.

### Obtention of Crude Extracts of Said Microalgae

The extract of P. tricornutum obtained according to one or other of the methods described below is also referred to as “crude extract” or “dry extract” according to the invention, as distinct from the oily extract described below corresponding to the crude (or dry) extract dispersed in an oil.

The biomass obtained from the culture can be used fresh or frozen and/or dried prior to preparation of the extract used according to the invention. It can be concentrated by removing all or part of the water using processes such as centrifugation, filtration, flocculation, sedimentation, coupled, or not, with drying steps by freeze-drying, vacuum drying, drum drying, atomization or any process enabling the water content of the biomass to be lowered. Cell lysis processes can then be implemented, such as the application of pressure, electrical flux, shear forces, the use of enzymes, or any other process enabling the destructuring of tissues, organs, cells or organelles.

By “dry biomass” it is meant dehydrated microalgal biomass containing less than 15%, advantageously less than 10%, even more advantageously less than 5% water. Preferably according to the invention, the biomass obtained after cultivation is frozen before drying. The biomass may first be centrifuged and then filtered to remove residual water before extraction.

In one embodiment of the invention, biomass extraction is a solid-liquid extraction in the presence of a solvent or solvent mixture selected from water, acetone, hexane, ethyl acetate, methyltetrahydrofuran, 2-methyloxolane, heptane, an alcohol selected from ethanol, methanol, isopropanol, a natural or synthetic oil or a mixture of natural or synthetic oils, methyltetrahydrofuran, 2-methyloxolane, heptane, an alcohol chosen from ethanol, methanol, isopropanol, a natural or branched oil, a glycol, a polyol, an alcohol/water or glycol/water mixture in a proportion of 99/1 to 1/99 (w/v).

Solid-liquid extraction of biomass according to the invention is generally carried out using a quantity of biomass of 0.1 to 20% by weight, advantageously from 1% to 20%, very advantageously from 5% to 15%, and even more advantageously from 10% by weight relative to the total volume of biomass and extraction solvent.

Extraction can be carried out at a temperature ranging from 4 °C to 300 °C, including ambient temperature, i.e. 20 °C. It is advantageously carried out at a temperature of between 20 °C and 50 °C, and even more advantageously at 30 °C.

According to a first embodiment of the invention, biomass extraction is carried out in water under subcritical conditions, at a temperature ranging from 100 °C to 300 °C, advantageously from 120 °C to 250 °C, even more advantageously at 120 °C. Extraction can be carried out at a given temperature or at successive increasing temperatures. In an advantageous embodiment of the invention, extraction is carried out at a temperature of 120 °C.

Alternatively, it can be carried out at a gradient of three increasing temperatures between 100 °C and 200 °C, such as 120 °C, 140 °C then 160 °C or 110 °C, 130 °C then 150 °C, or 120 °C, 145 °C then 170 °C.

Extraction under “subcritical conditions” means extraction in the presence of water, under conditions of temperature above 100 °C and pressure below 221 bar, the water remaining in the liquid state but having a lower viscosity and surface tension than water at room temperature, increasing its dielectric constant. Thus, the extraction pressure is comprised between 150 bar and 250 bar, preferably between 200 bar and 221 bar, advantageously in a pressurized extraction autoclave.

According to another embodiment of the invention, the biomass extraction is carried out by supercritical CO2 extraction. In this case, said extraction may optionally be carried out in the presence of a co-solvent chosen from ethanol, acetone, methanol or any combination thereof. Alternatively, the extraction with supercritical CO2 can be carried out alone without the presence of a co-solvent and followed by a further extraction with a polar solvent chosen from ethanol, acetone, methanol, hexane, heptane, advantageously ethanol.

Advantageously in the context of the invention, supercritical CO2 extraction is carried out in the presence of ethanol (10%) as co-solvent. The supercritical CO2 extraction conditions are such that the pressure measured in bar is greater than 74 bars, advantageously greater than 100 bars, even more advantageously 150 bars. The temperature is greater than 35 °C, advantageously greater than 45 °C, still advantageously 50 °C.

According to a particular and preferred embodiment of the invention, the extract of P. tricornutum is obtained by extraction in an alcohol/water mixture, in a proportion of 99/1 to 1/99 (w/v), advantageously still in a proportion of 95/5 to 90/10 (w/v). This is known as ‘solid-liquid’ extraction.

According to a particularly preferred embodiment, the crude extract is obtained by extraction in a 90/10 (w/v) ethanol/water mixture.

Thus, in a preferred mode of the invention, the crude extract is obtained from the frozen then dried biomass of P. tricornutum, by solid-liquid extraction in a 90/10 (w/v) ethanol/water mixture at 30 °C. The amount of biomass used for extraction is 10% by weight, based on the total weight of biomass and ethanol/water mixture. The crude extract obtained is decolorized with activated carbon and then filtered under the conditions described in example 1a). The residual ethanol is evaporated, then the extract can be used as such (“raw extract”) in powder form or dispersed in an oil (referred to as “oily extract” or “oily preparation” according to the invention) or incorporated into a final cosmetic composition (incorporation of the raw extract or advantageously the oily extract into the final cosmetic composition). According to another preferred embodiment of the invention, the crude extract is obtained from the frozen, then dried biomass of P. tricornutum, by supercritical CO2 extraction at a temperature of 50 °C and a pressure of 150 bar in the presence of ethanol as co-solvent (10%). The extract obtained is rinsed with water and then filtered under the conditions described in Example 1b).

The extract according to the invention can be used as such (raw extract) in powder form or dispersed in an oil (referred to as an “oily preparation” or “oily extract” according to the invention), or incorporated into a final cosmetic composition (incorporation of the raw extract or advantageously of the oily extract into the final cosmetic composition) with a “cosmetically acceptable excipient”, i.e. an excipient which is non-irritant to the skin or lips and non-toxic. The excipient is advantageously chosen from natural oils, in particular vegetable oils, such as argan oil, shea oil, jojoba oil, avocado oil, sweet almond oil, or any oil extracted from a plant or algae, including microalgae, notably selected from olive oil, rapeseed oil, linseed oil, sunflower oil, medium-chain triglyceride (MCT) oil, additional oils such as C11-C13 alkanes, ester oils and mixtures thereof, preservatives, emulsifiers, emollients, surfactants, moisturizers, thickeners, texture agents, conditioners, shine agents, texture agents, film-forming agents, pigments, dyes, fragrances, biological additives, chelating agents, astringents, polymers, reducing agents, pH regulating agents, humectants, conditioning agents, and mixtures thereof.

### Oily Preparation (Otherwise Known as an “Oily Extract” According to the Invention)

According to a particular mode, the extract used according to the invention is dispersed in at least one vegetable oil to form an “oily preparation” (or “oily extract”), which can be used as such or constitute an intermediate preparation of a final composition comprising other additional ingredients.

In particular, the extract used according to the invention can be dispersed in any oil extracted from a plant or an alga, including a microalgae, notably chosen from olive oil, rapeseed oil, linseed oil, sunflower oil, a medium chain triglyceride (MCT) oil. Medium Chain Triglycerides (MCT) are esters of glycerol and saturated fatty acids, with a hydrocarbon chain of 6 to 12 carbon atoms. A MCT oil can thus be chosen from coconut oil, advantageously coconut oil, palm kernel oil and palm oil, but can also be obtained from other fats or oils. Advantageously for the purposes of the invention, the vegetable oil is a MCT oil, very advantageously coconut oil.

According to a particular mode, said oil is present in a content by weight ranging from 50 to 99.8% by weight with respect to the total weight of the composition, advantageously from 75% to 99.5%, very advantageously in a content ranging from 97.5% to 99.4%, still advantageously ranging from 98% to 99% by weight with respect to the total weight of the composition, otherwise called oily preparation when the oil content represents from 97.5% to 99.4% by weight with respect to the total weight of the composition. This oily preparation can be used as is or as an intermediate preparation for a nutraceutical composition or a cosmetic composition further comprising other cosmetic ingredients and excipients such as those described above.

Thus, in a preferred embodiment of the invention, the oily preparation comprises coconut oil. According to a particular and preferred mode, to the crude extract obtained according to examples 1a) or 1b), a quantity of coconut oil ranging from 97.5 to 99.4% by weight is added, advantageously ranging from 98 to 99% by weight with respect to the total weight of the oily preparation.

According to a particular and preferred mode, said oily preparation advantageously further comprises an antioxidant and preferably vitamin E in a content ranging from 0.1% to 0.5% by weight relative to the total weight of the oily preparation. Advantageously, vitamin E is present in a content of 0.25% by weight relative to the total weight of the oily preparation. By “vitamin E” it is meant a tocopherol selected from δ-tocopherol, γ-tocopherol, β-tocopherol or δ-tocopherol, or a tocotrienol selected from α-tocotrienol, β-tocotrienol, γ-tocotrienol or δ-tocotrienol. Advantageously, this is α-tocopherol.

Thus, according to a particular and preferred mode, the extract is in the form of an oily extract comprising (i) from 0.5 to 2% by weight, preferably 1.5% by weight of an extract of P. tricornutum relative to the total weight of the oily extract, preferably an extract as defined in claim 3 or 4 (ii) from 97.5 to 99.4%, preferably from 98 to 99% by weight of a vegetable oil based on the total weight of the oily extract, preferably a medium-chain triglyceride (MCT) oil and even more preferably a coconut oil and (iii) from 0.1% to 0.5% by weight based on the total weight of the oily extract, preferably from 0.25% by weight of an antioxidant, preferably vitamin E. In particular, the oily extract comprises from 0.5 to 2% by weight, preferably 1.5% by weight of an extract of P. tricornutum prepared according to example 1a).

This oily preparation (or oily extract) can be used as such or as an intermediate composition in the preparation of a final composition.

### Final Composition

By “final composition” according to the invention, it is meant a composition comprising a) the crude (or dry) extract, or advantageously the oily extract according to the invention, consisting of at least one vegetable oil such as those described above and in particular coconut oil, and advantageously in addition an antioxidant in particular vitamin E, and b) at least one cosmetically acceptable excipient and one or more additional ingredients chosen from additional oils such as vegetable oils notably chosen from argan oil, shea oil, jojoba oil, avocado oil, sweet almond oil, or any oil extracted from a plant or algae, including microalgae, notably selected from olive oil, rapeseed oil, linseed oil, sunflower oil, medium-chain triglyceride (MCT) oil, C11-C13 alkanes, ester oils and mixtures thereof, preservatives, emulsifiers, emollients, surfactants, moisturizers, thickeners, texture agents, conditioners, shine agents, texture agents, film-forming agents, pigments, colorants, fragrances, biological additives, chelating agents, astringent agents, polymers, reducing agents, pH regulating agents, humectants, conditioning agents, and mixtures thereof.

The composition can also include any active cosmetic ingredient with the same effect of maintaining and/or increasing the volume of the skin and/or lips, such as hyaluronic acid, or a complementary effect, in particular an anti-aging, anti-wrinkle or skin firmness-enhancing ingredient, an ingredient active on cell renewal, a so-called anti-pollution ingredient, an active lightening ingredient or an ingredient active on the uniformity of skin tone, and mixtures thereof.

The extract used according to the invention may also be combined with one or more other algae or microalgae extracts, including an aqueous extract or lipophilic extract of Porphyridium cruentum, advantageously as an active anti-aging ingredient and protector against the combined effects of UV rays and pollution; and/or a hydrolysate of a coproduct extract of P. tricornutum, particularly useful for exfoliating the skin and/or reducing skin microrelief, and advantageously a hydrolysate of a coproduct of an ethanolic extract of P. tricornutum and/or an extract of the microalgae Tisochrysis lutea, in particular as an active ingredient to improve the comfort of sensitive skin.

The extract according to the invention can also be combined with an extract of the microalgae Dunaliella salina, in particular a hydroalcoholic extract, the alcohol being advantageously a C1-C6 alcohol, still advantageously ethanol, or a supercritical CO2 extract of the microalgae Dunaliella salina.

According to a particular and preferred mode, the extract of P. tricornutum according to the invention is considered to be effective for increasing the volume of the skin and/or lips when this increase is at least 1%, advantageously at least 2% and very advantageously at least 4% in the presence of said extract in vivo, compared to the volume of the skin or lips evaluated in vivo without addition of extract. This increase in skin volume is assessed in particular by taking lateral 3D photographs of faces at cheek level, analyzed by specific image analysis techniques well known to the skilled person.

Thus, a content of the extract P. tricornutum according to the invention, preferably of the oily extract according to the invention, will advantageously be used in a final composition of the invention, ranging from 0.1 to 10% by weight, preferably from 0.5 to 5% by weight, in particular from 0.5 to 3% by weight, advantageously from 0.75% to 2% by weight, still advantageously from 1% to 1.5% by weight and in particular from 1.5% by weight relative to the total weight of the final composition.

Even more preferably, the oily extract is present in a final composition of the invention in a content ranging from 1% to 1.5%, and preferably 1.5% by weight relative to the total weight of the final composition.

According to a particular and preferred mode, the oily extract according to the invention comprises an extract of P. tricornutum dispersed in a coconut oil and comprising vitamin E, preferably in the following proportions: (i) from 0.5 to 2% by weight with respect to the total weight of the oily extract, preferably 1.5% by weight, of an extract of P. tricornutum, in particular the one prepared according to example 1a), (ii) from 97.5 to 99.4%, preferably 98 to 99% by weight, based on the total weight of the oil extract, of an MCT oil, in particular coconut oil, and (iii) 0.1% to 0.5%, preferably 0.25% by weight, based on the total weight of the oil extract, of an antioxidant, preferably vitamin E.

Even more preferably, the oily extract comprises (i) 1.5% by weight of a P. tricornutum extract, (ii) 98 to 99% by weight of a coconut oil and (iii) 0.25% by weight of vitamin E based on the total weight of the oily extract. This results in a content of dry extract (active ingredient) of P. tricornutum in the final composition ranging from 0.0015 to 0.15% by weight, preferably from 0.0075 to 0.075% by weight, in particular from 0, 0075 to 0.045% by weight, advantageously from 0.01125% to 0.03% by weight, even more advantageously from 0.015% to 0.0225% by weight and in particular to 0.00225% by weight relative to the total weight of the final composition.

According to a preferred mode, the oily extract of P. tricornutum prepared according to example 1a) is used in a content ranging from 0.5 to 2% by weight, preferably 1.5% by weight relative to the total weight of the composition (oily preparation or final composition). This results in a content of P. tricornutum dry extract (active ingredient) in the final composition ranging from 0.015 to 0.03% by weight, preferably 0.0225% by weight and in particular to 0.00225% by weight based on the total weight of the final composition.

### Galenic

The cosmetic composition for topical application onto the skin and/or lips can be under the form of a powder, cream, serum, soap, shower gel, oily solution, oil-in-water emulsion or water-in-oil emulsion, mask, lotion, mousse, lipstick or lip balm. The composition can also be under the form of a patch or be encapsulated in a form chosen from spheres, microspheres or liposomes. Advantageously, the composition is in the form of a cream or serum.

When the composition is intended for oral administration as a nutraceutical, it may be under powder form, in which case it is available as capsules, tablets, lozenges, liquid or loose powder.

The cosmetic composition is useful for maintaining and/or increasing the amount of hyaluronic acid, for maintaining and/or increasing the volume of the skin and/or lips. The cosmetic composition has a plumping effect on the skin, advantageously on the skin of the face and lips, enables hollow areas of the face to be filled in, and can be used for this purpose.

### Method of Cosmetic Care

Another object relates to a method of cosmetic care to maintain and/or increase the volume of the skin and/or lips, and/or to plump the skin and/or lips, and/or to fill hollow areas of the face, comprising the oral administration or the topical application, preferably the topical application, of an extract of P. tricornutum as defined according to the invention, preferably an oily extract as defined above or a cosmetic composition comprising it.

In particular, the method according to the invention is intended for people presenting volume loss in the central region of the face, such as people aged at least 40, in particular at least 50, or even at least 60, or slimmed people. In particular, these people can present flattened cheekbones, sunken cheeks and dark circles, a more pronounced nasolabial fold, sunken temples and/or thinned lips. Particularly in the case of people with facial hollows, the aim is to fill in these hollow areas to reshape the face and add volume to the eyes, for a more youthful, less tired appearance.

In an advantageous embodiment of the invention, the method of cosmetic care comprises the topical application of the extract according to the invention or of a cosmetic composition comprising it onto at least one area of skin, advantageously healthy skin, and/or at least one area of lip, advantageously healthy, the area of skin being chosen from any area of the face and/or any area of the body chosen from arms, including forearms, legs, thighs, back, torso, chest, neck, decollete, buttocks, feet and/or hands, advantageously on any hollow area of facial skin chosen from cheeks, cheekbones, forehead, nose contour, nasolabial fold, temples, eye contour, chin, mouth contour and/or lips, preferably cheeks, cheekbones, nasolabial fold, temples, eye contour, mouth contour and/or lips.

The extract used in the method of cosmetic is as defined above.

In particular, an oily extract or a final composition comprising it will be used, such as described above.

In an advantageous embodiment, the crude (or dry) extract of P. tricornutum is as defined in the present invention. Preferably, it is a crude extract obtained in an alcohol/water mixture, in a proportion of 99/1 to 1/99 (w/v), advantageously still in a proportion of 95/5 to 90/10 (w/v), very advantageously in an ethanol/water mixture in a proportion of 90/10 (w/v).

According to a particular and preferred mode, the oily extract according to the invention comprising an extract of P. tricornutum dispersed in a coconut oil and comprising vitamin E, will be used, preferably in the following proportions: (i) from 0.5 to 2% by weight, preferably 1.5% by weight of an extract of P. tricornutum with respect to the total weight of the oily extract, in particular that prepared according to example 1a), (ii) from 97.5 to 99.4%, preferably 98 to 99%, by weight, based on the total weight of the oil extract, of an MCT oil, in particular coconut oil, and (iii) 0.1% to 0.5%, preferably 0.25%, by weight, based on the total weight of the oily extract, of an antioxidant, preferably vitamin E.

Even more preferably, the oily extract comprises (i) 1.5% by weight of an extract of P. tricornutum, (ii) 98 to 99% by weight of a coconut oil and (iii) 0.25% by weight of vitamin E based on the total weight of the oily extract.

According to another particular mode, a final composition will be used comprising the oily extract according to the invention and one or more other additional ingredients as described above, in particular additional oils.

The crude (or dry) extract is even more preferably obtained from a frozen biomass of P. tricornutum. A final composition of the invention will advantageously contain an oily extract of P. tricornutum ranging from 0.1 to 10% by weight, preferably from 0.5 to 5% by weight, in particular from 0.5 to 3% by weight, advantageously from 0.75% to 2% by weight, even more advantageously from 1% to 1.5% by weight and in particular to 1.5% by weight relative to the total weight of the final composition.

According to a preferred method, the oily extract of P. tricornutum prepared according to example 1a) is used in a content ranging from 0.5 to 2% by weight, preferably 1.5% by weight relative to the total weight of the composition (oily preparation or final composition).

According to a particular and preferred mode, the composition is a face serum or lip balm, in particular a face serum comprising an extract of P. tricornutum as defined according to the invention, preferably the oily extract according to the invention, in a content ranging from 0.1 to 10% by weight, preferably from 0.5 to 5% by weight, in particular from 0.5 to 3% by weight, advantageously from 0.75% to 2% by weight, still advantageously from 1% to 1.5% by weight and in particular to 1.5% by weight relative to the total weight of the final composition.

### Other Uses

A final object relates to an extract of P. tricornutum or a dermatological or pharmaceutical composition comprising it for use in promoting skin healing, in particular by maintaining and/or increasing the amount of hyaluronic acid at the level of the fundamental substance of the skin dermis.

Thus, the invention also relates to a P. tricornutum extract or dermatological or pharmaceutical composition comprising it for use in promoting healing of the skin, in particular of people with damaged skin and/or damaged lips, wounds, lesions and/or superficial wounds.

The dermatological or pharmaceutical composition further comprises at least one dermatologically or pharmaceutically acceptable excipient.

The extract used is as defined above. In particular, an oily extract or a final composition comprising it, as described above, is used.

In an advantageous embodiment, the crude (or dry) extract of P. tricornutum is such as defined in the present invention. Preferably, it is a crude extract obtained in an alcohol/water mixture, in a proportion of 99/1 to 1/99 (w/v), advantageously still in a proportion of 95/5 to 90/10 (w/v), very advantageously in an ethanol/water mixture in a proportion of 90/10 (w/v).

According to a particular and preferred mode, the oily extract according to the invention comprising an extract of P. tricornutum dispersed in a coconut oil and comprising vitamin E will be used, preferentially in the following proportions: (i) from 0.5 to 2% by weight, preferentially 1.5% by weight relative to the total weight of the oily extract of an extract of P. tricornutum, in particular the one prepared according to example 1a), (ii) from 97.5 to 99.4%, preferably 98 to 99% by weight based on the total weight of the oily extract, of an MCT oil, in particular coconut oil, and (iii) 0.1% to 0.5%, preferably 0.25% by weight of an antioxidant, preferably vitamin E.

Even more preferably, the oily extract comprises (i) 1.5% by weight of an extract of P. tricornutum, (ii) 98 to 99% by weight of a coconut oil and (iii) 0.25% by weight of vitamin E based on the total weight of the oily extract.

According to another particular mode, a final composition will be used comprising the oily extract according to the invention and one or more other additional ingredients as described above, in particular additional oils.

The crude (or dry) extract is even more preferably obtained from a frozen biomass of P. tricornutum. A final composition of the invention will advantageously contain an oily extract of P. tricornutum ranging from 0.1 to 10% by weight, preferably from 0.5 to 5% by weight, in particular from 0.5 to 3% by weight, advantageously from 0.75% to 2% by weight, even more advantageously from 1% to 1.5% by weight and in particular to 1.5% by weight relative to the total weight of the final composition.

According to a preferred method, the oily extract of P. tricornutum prepared according to example 1a) is used in a content ranging from 0.5 to 2% by weight, preferably 1.5% by weight relative to the total weight of the composition (oily preparation or final composition).

The pharmaceutical or dermatological composition further comprises at least one vegetable oil as defined in the invention, advantageously an MCT oil, very advantageously coconut oil. Said oil may be incorporated in a content by weight of 50 to 99.8% relative to the total weight of the composition, advantageously in a content ranging from 97.5% to 99.4%, even more advantageously from 98% to 99% by weight relative to the total weight of the composition, otherwise called oily preparation (or oily extract) for an oil content in the composition ranging from 97.5% to 99.4% by weight relative to the total weight of the composition.

According to a particular mode, a pharmaceutical or dermatological composition comprising the extract as defined according to the invention is used, preferably the oily extract according to the invention, or a final pharmaceutical or dermatological composition further comprising at least one additional ingredient chosen from preservatives, biocidal agents, antimicrobial agents, emulsifiers, emollients, surfactants, moisturizers, thickeners, texture agents, conditioners, shine agents, film-forming agents, pigments, colorants, fragrances, chelating agents, polymers, reducing agents, pH-regulating agents, humectants, conditioning agents, and mixtures thereof, can be used. Phaeodactylum tricornutum extract or a pharmaceutical or dermatological composition comprising it may thus be intended for people with cuts, wounds or other skin lesions, to promote healing of the skin and/or lips.

The following examples refer to the description. These examples are illustrative and do not limit the scope of the invention.

Unless otherwise stated, temperature is expressed in degrees Celsius (°C), pressure in bar, percentages are given in weight/weight (w/w). Ppm stands for Part Per Million (0.01%=100 ppm).

### Example 1: Modes of Preparation of an Extract of Phaeodactylum tricornutum and an Oily Preparation (Oily Extract) Comprising it

### Ex. 1a) Hydro-Alcoholic Extraction (Crude Extract 1a)

A quantity of 10% by weight relative to the total weight of biomass and solvent, of frozen and dried biomass from an auxotrophic culture in photobioreactor of a P. tricornutum strain isolated, domesticated and grown in France, was extracted in the presence of an ethanol/water mixture 90/10 (w/v), at a temperature of 30 °C. The extract obtained was rinsed with water, then decolorized with activated charcoal, and filtered down to 1 μm. The extract obtained is an extract containing fatty acids and corresponds to the extract according to the invention.

### Ex. 1b) Supercritical CO₂ Extraction (Crude Extract 1b)

In another preferred embodiment, the extract is obtained from the same frozen and dried P. tricornutum biomass as described in example 1a) but by extraction with supercritical CO2 at a temperature of 50 °C and a pressure of 150 bar in the presence of ethanol as co-solvent (10%). The extract obtained was rinsed with water and then filtered under the conditions described in example 1a).

Coconut oil (MCT oil) was then added to the crude extract obtained in examples 1a) or 1b) respectively, in a quantity of between 98 and 99% (w/w). The whole was homogenized and the residual ethanol evaporated. A final amount of 0.25% by weight of the total composition of α-tocopherol was added. Phaeodactylum tricornutum extract is present in this oily preparation (oily extract) in an amount of 1.5% by weight based on the total weight of said oily preparation.

This oil extract is then used in the following examples.

### Example 2: In Vitro Plumping Effect of the Extract of P. tricornutum: Increase in Skin Volume

### Protocol:

Fibroblasts and adipocytes derived from healthy (non-pathological) human skin from healthy donors were co-cultured after seeding in DMEM (Dulbecco's Modified Eagle Medium) in the presence of Fetal Bovine Serum (FBS) (10%) for a period of 24 hours. A final concentration by weight relative to the total volume of the culture of 0, 1% (w/v) or 0.05% (w/v) of the oily preparation comprising the extract as prepared according to example 1a) was added to the culture, which was then run for a further 24 hours after addition of the extract. The same culture without extract (DMEM culture medium) was run under the same conditions. Triton (2%) was added to the cultures to induce cell lysis. The culture medium was collected and the fibroblasts fixed with paraformaldehyde solution (4%). Each condition was performed in triplicate.

The protein content of hyaluronic acid was measured using the ELISA immunoassay technique.

The results are presented in Table 1 below, and correspond to the average of the 3 samples analyzed, with the quantity of hyaluronic acid measured after 24 hours of culture expressed as a % relative to the control (without extract) and related to the number of fibroblasts.

Results: Average quantity of hyaluronic acid (HA) produced.

TABLE 1

| | Average | Standard error |
|---|---|---|
| | [HA] | of mean |
| Control (without extract) | 100 | 19.1 |
| Oily extract of P. tricornutum according | 242.1 | 17.8 |
| to ex. 1a) at 0.1% (w/w) | | |
| Oily extract of P. tricornutum according | 135.5 | 0.5 |
| to ex. 1a) at 0.05% (w/w) | | |

Conclusion: the oily extract according to the invention increased the quantity of hyaluronic acid measured in fibroblast cultures by at least 15% (taking into account the standard error of mean and up to 35% without taking it into account), and up to more than 140%, with a noticeable dose effect, and presented a greater effect than that of the control, demonstrating its efficacy in increasing skin volume, and therefore its plumping effect.

### Example 3: Plumping Serum for the Face

The formulation below is a plumping serum for the face. The formulation is prepared by mixing the various ingredients using techniques well known to the one skilled in the art until a homogeneous serum is obtained. Percentages are expressed by weight in relation to the total weight of the serum (w/w).

TABLE 2

| Ingredients (INCI name) | % by weight |
|---|---|
| Undecane and tridecane | Qsp 100 |
| Ether dicaprylyl | 27.00 |
| Coconut alkanes & coco-caprylate/caprate | 27.00 |
| Oily preparation comprising the extract of | 1.5 (ie. 0.00225% of extract = |
| P. tricornutum according to example 1a) | active material) |
| Fragance | 0.55 |

This oily serum was evaluated in a 28-day clinical study involving a population of 30 Caucasian women aged between 45 and 70, with healthy skin as defined by the invention, and in particular hollowed skin areas (cheeks, cheekbones, nasolabial fold, eye contour, etc.). Each woman had the above-described oily serum applied to a first half-face, and a placebo composition without the extract according to the invention applied to the second half-face. Each serum was advantageously applied twice a day, morning and evening, to each hemi-face, including the nose and temples.

The improvement in skin volume was assessed by taking lateral 3D photographs of the faces at cheek level, analyzed by specific image analysis techniques well known to those skilled in the art.

The results of the clinical study showed an increase in the volume of the skin on the cheeks treated with the serum comprising the extract according to the invention, compared with the hemi-face treated with the placebo. The extract according to the invention is therefore effective in increasing the volume of the skin, and preferably the skin of the face and hollowed areas such as the cheeks, cheekbones and eye contour. It is also effective for increasing lip volume. It is a plumping active extract.

1.-14. (canceled)

15. Method of cosmetic care comprising the topical application to a person in need thereof of an effective amount of an extract of Phaeodactylum tricornutum or of a cosmetic composition comprising it to maintain and/or increase the volume of healthy skin and/or healthy lips, and/or to plump healthy skin and/or healthy lips, and/or to fill in hollow areas of the face.

16. Method according to claim 15, wherein the extract of Phaeodactylum tricornutum is obtained in an alcohol/water mixture, in a proportion of 99/1 to 1/99 (w/v).

17. Method according to claim 16, wherein the extract of Phaeodactylum tricornutum is obtained in an alcohol/water mixture, in a proportion of 95/5 to 90/10 (w/v).

18. Method according to claim 17, wherein the extract of Phaeodactylum tricornutum is obtained in an ethanol/water mixture in a proportion of 90/10 (w/v).

19. Method according to claim 15, wherein the extract is in the form of an oily extract comprising (i) from 0.5 to 2% by weight of an extract of Phaeodactylum tricornutum relative to the total weight of the oily extract, (ii) from 97.5 to 99.4% of a vegetable oil based on the total weight of the oily extract, and (iii) from 0.1% to 0.5% by weight based on the total weight of the oily extract of an antioxidant.

20. Method according to claim 19, wherein the extract is in the form of an oily extract comprising (i) 1.5% by weight of an extract of Phaeodactylum tricornutum, (ii) 98 to 99% by weight of a medium-chain triglyceride (MCT) oil, and (iii) 0.25% by weight of vitamin E based on the total weight of the oily extract.

21. Method according to claim 20, wherein the medium-chain triglyceride (MCT) oil is coconut oil.

22. Method according to claim 19, wherein the oily extract is present in a final cosmetic composition in a content ranging from 0.1 to 10% by weight.

23. Method according to claim 22, wherein the oily extract is present in a final cosmetic composition in a content ranging from 0.5 to 5% by weight.

24. Method according to claim 23, wherein the oily extract is present in a final cosmetic composition in a content of 1.5% by weight relative to the total weight of the final composition.

25. Method according to claim 22, wherein the cosmetic composition comprises at least one cosmetically acceptable excipient and one or more additional ingredients chosen from additional oils such as vegetable oils chosen in particular from argan oil, shea oil, jojoba oil, avocado oil, sweet almond oil, or any oil extracted from a plant or algae, including microalgae, in particular chosen from olive oil, rapeseed oil, linseed oil, sunflower oil, medium-chain triglyceride (MCT) oil, C11-C13 alkanes, ester oils and mixtures thereof, preservatives, emulsifiers, emollients, surfactants, moisturizers, thickeners, texture agents, conditioners, shine agents, texture agents, film-forming agents, pigments, colorants, fragrances, biological additives, chelating agents, astringent agents, polymers, reducing agents, pH regulating agents, humectants, conditioning agents, and mixtures thereof.

26. Method of cosmetic care according to claim 15, comprising the topical application of the extract of Phaeodactylum tricornutum or of the cosmetic composition comprising it onto any area of the face and/or any area of the body chosen from arms, including forearms, legs, thighs, back, torso, chest, neck, décolleté, buttocks, feet and/or hands, advantageously on any hollow area of facial skin selected from cheeks, cheekbones, forehead, nose contour, nasolabial fold, temples, eye contour, chin, mouth contour and/or lips, preferably cheeks, cheekbones, nasolabial fold, temples, eye contour, mouth contour and/or lips.

27. Method of cosmetic care according to claim 26, wherein the composition is in the form of a powder, cream, serum, soap, shower gel, oily solution, oil-in-water emulsion, water-in-oil emulsion, mask, lotion, mousse, lipstick, or lip balm.

28. Method for promoting healing of the skin of persons with damaged skin and/or damaged lips, wounds, lesions and/or superficial wounds, comprising the topical application of an extract of Phaeodactylum tricornutum or a dermatological or pharmaceutical composition comprising it.

29. Method of cosmetic care comprising the oral administration to a person in need thereof of an effective amount of an extract of Phaeodactylum tricornutum to maintain and/or increase the volume of healthy skin and/or healthy lips, and/or to plump healthy skin and/or healthy lips, and/or to fill in hollow areas of the face.

30. Method according to claim 29, wherein the extract of Phaeodactylum tricornutum is obtained in an alcohol/water mixture, in a proportion of 99/1 to 1/99 (w/v).

31. Method according to claim 30, wherein the extract of Phaeodactylum tricornutum is obtained in an ethanol/water mixture in a proportion of 90/10 (w/v).

32. Method according to claim 29, wherein the extract is in the form of an oily extract comprising an extract of Phaeodactylum tricornutum dispersed in coconut oil and vitamin E.
